(19) United States
(12) Patent Application Publication
(10) Pub. No.: US 2024/0423714 A1
(43) Pub. Date: Dec. 26, 2024

(54) INTEGRATED LASER BASKET (ILB) AND METHOD OF USING THE SAME

(71) Applicant: Daniel Rabinowitz, Cresskill, NJ (US)

(72) Inventor: Daniel Rabinowitz, Cresskill, NJ (US)

(21) Appl. No.: 18/510,507

(22) Filed: Nov. 15, 2023

Related U.S. Application Data
(63) Continuation of application No. 18/340,027, filed on Jun. 23, 2023.

(51) Int. Cl. A61B 18/26

(52) U.S. Cl. CPC A61B 18/26; A61B 2017/00867; A61B 2018/00267; A61B 2018/2222

(57) ABSTRACT

The Integrated Laser Basket (ILB)—a medical device used for treating various types of stone deposits, including, but not limited to, kidney stones, gallstones, bladder stones, prostate stones, pancreatic stones, or any combination thereof, in human or animal patients. The ILB merges the benefits of laser lithotripsy and stone retrieval in a single device, offering a more comprehensive and minimally invasive approach to stone management. Example methods of using the ILB to treat various types of stone deposits in human and non-human patients are also described.

### RELATED APPLICATIONS

The present application is a continuation of U.S. patent application Ser. No. 18/340,027 filed on Jun. 23, 2023, the contents of which are incorporated in their respective entirety for all purposes.

### FIELD

The field of the present disclosure relates generally to medical devices, and more specifically to a compact integrated laser basket designed for the treatment and removal of various types of stone deposits in patients.

### BACKGROUND

Medical baskets are used for treating and removing stone deposits in patients. These stones may include, but are not limited to, kidney stones, gallstones, bladder stones, prostate stones, and pancreatic stones. Existing devices often have limitations due to their size and flexibility, making it challenging to treat stone deposits in tight spaces or restricting the amount of fluid that can flow through the scope, which affects visibility during treatment. Furthermore, current laser fibers used for stone treatment have size limitations, further restricting the effectiveness and application of existing devices.

### SUMMARY

The present disclosure relates to an integrated laser basket (ILB) for treating and removing stone deposits in patients. The ILB may comprise a pattern of composite laser fibers, which can be integral structures including at least one thulium laser fiber; and a shape memory alloy. The ILB can be designed to treat stone deposits by emitting at least one laser beam from the integral structures toward the stone deposits.

The ILB may be suitable for treating various types of stone deposits, such as kidney stones, gallstones, bladder stones, prostate stones, pancreatic stones, or any combination thereof, and can be used in human or animal patients. The ILB can treat stone deposits without necessitating a waveguide, effectively rendering waveguides optional. In addition, the ILB does not require a holmium laser fiber, though at least one holmium laser fiber can be employed as an ancillary feature.

Example methods of using the ILB to treat various types of stone deposits in human and non-human patients are also included in the present disclosure.

Covered embodiments are defined by the claims, not this summary. This summary is a high-level overview of various aspects and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

### BRIEF DESCRIPTION OF THE DRAWINGS

FIGS. 1A-1E present perspective views of an embodiment of the integrated laser basket (ILB), as per the present disclosure, showcasing example arrangements of composite laser fibers.

FIGS. 2A-2B depict a schematic representation of a composite laser fiber, illustrating examples of the integration of the thulium laser fiber and the shape memory alloy.

FIG. 3 shows an example embodiment of the ILB in an open conformation, revealing the side fire capability of the laser fibers.

FIG. 4 exhibits an example embodiment of the ILB in a closed conformation, displaying the end fire functionality of the laser fibers.

Some embodiments of the present disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

### DETAILED DESCRIPTION

The Integrated Laser Basket (ILB) of the current disclosure can be characterized as a medical device designed to treat various types of stone deposits, including, but not limited to, kidney stones, gallstones, bladder stones, prostate stones, pancreatic stones, or any combination thereof, in human or animal patients. The ILB can the benefits of laser lithotripsy and stone retrieval in a single device, offering a more comprehensive and minimally invasive approach to stone management.

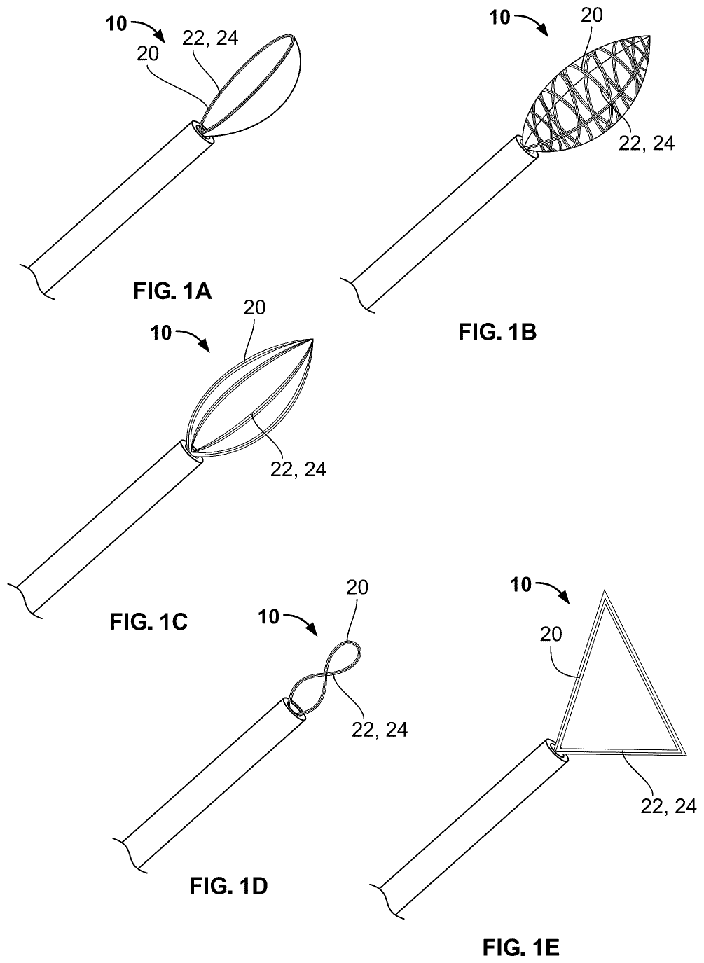

Referring to FIGS. 1A-1E, perspective views of example embodiments of an ILB 10 are shown, illustrating example patterns of the composite laser fibers 20. In one embodiment, the composite laser fibers 20 can be a mesh pattern (FIG. 1B-C), a helical pattern (FIG. 1D) a three-wire pattern (FIG. 1A), a four-wire pattern (not shown), a stone retrieval pattern (FIG. 1E), or any combination thereof.

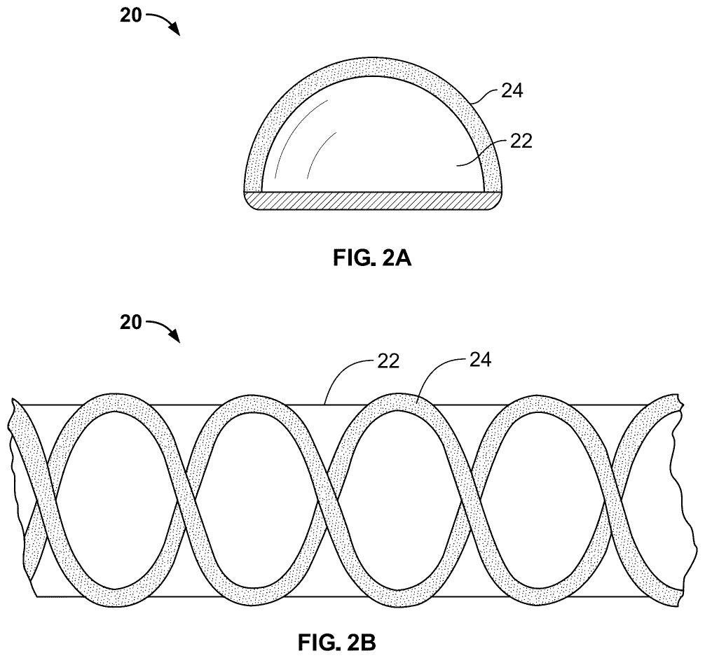

FIGS. 2A-2B display non-limiting examples of a composite laser fiber 20, demonstrating the integration of the thulium laser fiber 22 and the shape memory alloy 24. “Composite laser fibers,” as per the current disclosure, comprise a combination of a laser fiber (such as a thulium laser fiber 22) and a shape memory alloy 24, which is integrated (e.g., interwoven, coated, or through another integration mechanism) with the shape memory alloy 24. Given that the composite laser fibers 20 combine the thulium laser fiber 22 and the shape memory alloy 24 into a single structure, the composite laser fibers 20 can be conceptualized as “integral structures.”

The thulium laser fiber 22, like a holmium laser fiber, can emit laser energy in the infrared spectrum to effectively break down stone deposits while causing minimal damage to surrounding tissues. However, unlike a holmium laser fiber, the thulium laser fiber 22 is sufficiently small to allow the thulium laser fiber 22 to form integral structures with at least one shape memory alloy 24. For instance, the thulium laser fiber 22 may have a diameter ranging from 50 microns to 1000 microns, 100 microns to 1000 microns, 250 microns to 1000 microns, 500 microns to 1000 microns, 750 microns to 1000 microns, 50 microns to 750 microns, 50 microns to 500 microns, 50 microns to 100 microns, 100 microns to 750 microns, 250 microns to 500 microns, or any combination thereof, including all nested ranges. In certain examples, the ILB 10 does not require a holmium laser fiber, though at least one holmium laser fiber can be employed as an ancillary feature, such as but, not limited to, as an optional waveguide.

The presence of the shape memory alloy 24 in the composite laser fibers 20 allows the ILB 10 to maintain shape while offering flexible positioning during treatment. “Shape memory alloys,” such as shape memory alloy 24, are metal alloys with shape memory properties, enabling them to return to their original shape after deformation when heated or exposed to a specific temperature range. The shape memory alloy 24 may comprise any suitable shape memory material, such as, but not limited to, nitinol, Fe—Mn—Si, Cu—Zn—Al, Cu—Al—Ni, or any combination thereof.

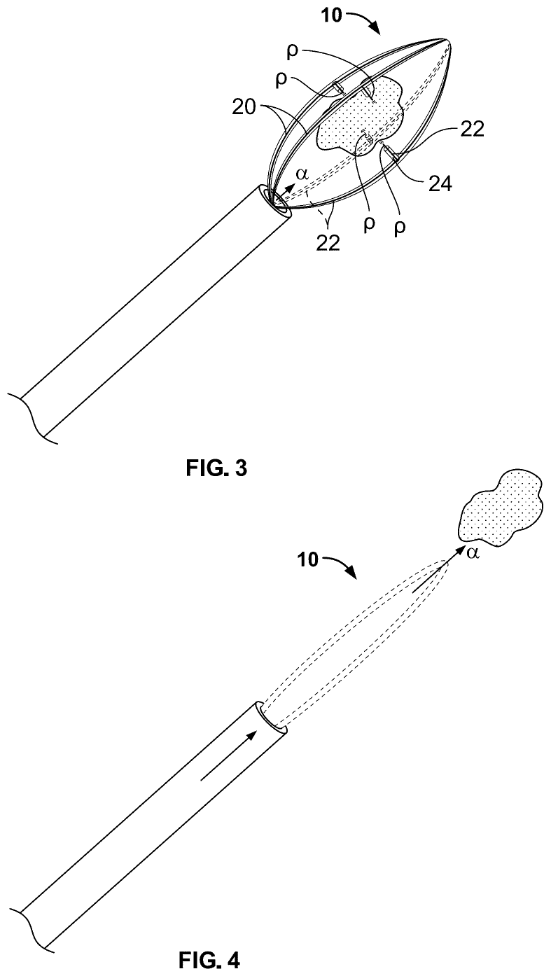

FIG. 3 presents an embodiment of the ILB 10 in an open conformation, displaying the side fire capability of the composite laser fibers 20, while FIG. 4 exhibits a top view of an embodiment of the ILB 10 in a closed conformation, demonstrating the end fire functionality of the laser fibers.

The ILB 10 can be configured to emit at least one laser beam towards the stone deposits in a radial direction p, an axial direction a, or any combination thereof. For instance, as shown in FIGS. 3-4, the radial direction p refers to the emission of laser beams inward towards the center of the ILB 10, using “side fire” capability to target stone deposits from multiple directions. The axial direction a refers to the emission of laser beams along the length of the ILB 10 using end fire functionality, which has traditionally been associated with an insertable waveguide. However, the ILB 10 of the present disclosure, while being compatible with an insertable waveguide, does not require an insertable waveguide due to the presence of the thulium laser fiber 22 within the integral structure of the composite laser fiber 20 being able to target stones from the radial direction p.

The firing of the laser beams from the from the radial direction p may be accomplished, in some embodiments, by having a portion of the thulium laser fiber 22 extend radially and inwardly toward the center of the basket. In embodiments where the integral structures are interwoven, the extending portion of the thulium laser fiber 22 may form a “loose thread” from the interwoven structure. In embodiments where the integral structures are coated, an uncoated portion of the thulium laser fiber 22 may extend radially and inwardly from the interwoven structure.

The ILB 10 can be introduced into the patient's body using a minimally invasive procedure, typically through a natural body orifice or a small incision. Once positioned at the treatment site (e.g., bladder, kidney, prostate, pancreas, gallbladder or other site), the ILB 10 can be adjusted to the desired conformation, either open @ or closed K, depending on the specific requirements of the procedure. The composite laser fibers 20, with their integrated thulium laser fiber 22 and shape memory alloy 24, enable targeted laser energy delivery in both radial p and axial a directions.

During the procedure, a physician can manipulate the ILB 10 to ensure optimal stone fragmentation and retrieval. The various configurations and basket styles, such as mesh 11, helical pattern 12, three-wire pattern 13, four-wire pattern 14, and stone retrieval pattern 15, provide adaptability and versatility to accommodate diverse treatment scenarios. After successful treatment, the ILB 10 can be easily retracted and removed from the patient's body, facilitating a faster recovery, and reduced post-operative complications.

The example ILB 10 of the present disclosure can provide a versatile and minimally invasive solution for treating various types of stone deposits in human and animal patients. By combining the advantages of laser lithotripsy and stone retrieval in a single device, the ILB 10 can offer enhanced efficiency and flexibility during treatment. The use of composite laser fibers 20, integrating thulium laser fiber 22 and shape memory alloy 24 can facilitate the formation of integral structures with various configurations and basket styles. This approach ensures accurate targeting of stone deposits while minimizing damage to surrounding tissues, ultimately improving patient outcomes and streamlining the overall treatment process.

The present disclosure also pertains to a method of treating stone deposits using the ILB. The method may include a step of obtaining the ILB. The method may also include a step of emitting laser beams from these integral structures towards the stone deposits, facilitating their fragmentation. This method can be used to treat a variety of stone deposits, including kidney stones, gallstones, bladder stones, prostate stones, pancreatic stones, or any combination thereof. It is also applicable to both human and animal patients.

The method may also involve a possibility of emitting the laser beams in a radial direction, an axial direction, or a combination of both. The method can be performed with or without a waveguide, emphasizing the method's adaptability. Moreover, the method does not require the use of a holmium laser fiber, which simplifies the method and enhances the usability of the ILB.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein. Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure, which are intended to be illustrative, and not restrictive.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

All prior patents, publications, and test methods referenced herein are incorporated by reference in their entireties.

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

Any feature or element that is positively identified in this description may also be specifically excluded as a feature or element of an embodiment of the present disclosure as defined in the claims.

As used herein, the term “consisting essentially of” limits the scope of a specific claim to the specified materials or steps and those that do not materially affect the basic and novel characteristic or characteristics of the specific claim.

The disclosure described herein may be practiced in the absence of any element or elements, limitation or limitations, which is not specifically disclosed herein. Thus, for example, in each instance herein, any of the terms “comprising,” “consisting essentially of and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

1. An integrated laser basket, where the integrated laser basket is configured to treat stone deposits in at least one patient, the integrated laser basket comprising:
a pattern of composite laser fibers, where the composite laser fibers are integral structures comprising:
at least one thulium laser fiber; and
a shape memory alloy;
where the integrated laser basket is configured to treat the stone deposits by emission of at least one laser beam, from the integral structures, toward the stone deposits.

2. The integrated laser basket of claim 1, where the pattern is a mesh, a helical pattern, a three-wire pattern, a four-wire pattern, stone retrieval pattern, or any combination thereof.

3. The integrated laser basket of claim 1, where the composite laser fibers are integral structures comprising:
the at least one thulium laser fiber having a coating comprising the shape memory alloy;
the at least one thulium laser fiber interwoven with the shape memory alloy; or
any combination thereof.

4. The integrated laser basket of claim 1, where the thulium laser fiber has a diameter ranging from 50 microns to 1000 microns.

5. The integrated laser basket of claim 1, where the shape memory alloy comprises: nitinol, Fe—Mn—Si, Cu—Zn—Al, Cu—Al—Ni, or any combination thereof.

6. The integrated laser basket of claim 1, where the basket is configured to emit at least one laser beam toward the stone deposits in a radial direction, an axial direction, or any combination thereof.

7. The integrated laser basket of claim 1, where the stone deposits comprise kidney stones, gallstones, bladder stones, prostate stones, pancreatic stones or any combination thereof.

8. The integrated laser basket of claim 1, where the at least one patient comprises a human patient.

9. The integrated laser basket of claim 1, where the integrated laser basket is configured to treat the stone deposits without an insertable waveguide.

10. The integrated laser basket of claim 1, where the integrated laser basket does not comprise a holmium laser fiber.

11. The integrated laser basket of claim 1, where the integrated laser basket further comprises a holmium laser fiber.

12. The integrated laser basket of claim 3, where a portion of the thulium laser fiber extends inwardly and radially from at least one of the integral structures to allow the at least one laser beam to be emitted toward the stone deposits in a radial direction.

13. The integrated laser basket of claim 3, where a portion of the thulium laser fiber is uncoated, with the uncoated portion extending radially from at least one of the integral structures to allow the at least one laser beam to be emitted toward the stone deposits in a radial direction.

14. A method of using an integrated laser basket to treat stone deposits in at least one patient, the method comprising:
obtaining the integrated laser basket, where the integrated laser basket comprises:
a pattern of composite laser fibers, where the composite laser fibers are integral structures comprising:
at least one thulium laser fiber; and
a shape memory alloy;
treating the stone deposits by emitting at least one laser beam, from the integral structures, toward the stone deposits.

15. The method of claim 14, treating the stone deposits comprises emitting the at least one laser beam toward the stone deposits in a radial direction, an axial direction, or any combination thereof.

16. The method of claim 14, where the stone deposits comprise kidney stones, gallstones, bladder stones, prostate stones, pancreatic stones, or any combination thereof.

17. The method of claim 14, where the at least one patient comprises a human patient.

18. (canceled)

19. The method of claim 14, where the method is performed with an insertable waveguide.

20. The method of claim 14, where the method does not include any steps that require a holmium laser fiber.
